(19) United States
(12) Patent Application Publication
(10) Pub. No.: US 2025/0243455 A1
(43) Pub. Date: Jul. 31, 2025

(54) METHODS FOR PRESERVING NEURAL PROGENITOR CELL SURVIVAL IN VITRO AND IN VIVO

(71) Applicant: BrainXell Therapeutics, Inc., Madison, WI (US)

(72) Inventors: Young Mook Lee, Madison, WI (US); Maria-Daniela Cirnaru, Madison, WI (US); Matthew Goodus, Madison, WI (US)

(21) Appl. No.: 19/041,741

(22) Filed: Jan. 30, 2025

Related U.S. Application Data
(60) Provisional application No. 63/627,583, filed on Jan. 31, 2024.

(51) Int. Cl. C12N 5/079; A61K 35/30

(52) U.S. Cl. CPC C12N 5/0618; A61K 35/30; C12N 2506/03; C12N 2513/00

(57) ABSTRACT

Disclosed herein are stable live-cell compositions comprising a plurality of spheroids derived from neural progenitor cells and methods of making the spheroids. Also disclosed herein are methods of treating a neuronal disorder or disease.

### CROSS-REFERENCE TO RELATED APPLICATIONS

This application claims priority to U.S. Provisional Patent Application No. 63/627,583, filed Jan. 31, 2024, which is incorporated herein by reference in its entirety.

### FIELD

This disclosure relates to stable live-cell compositions comprising a plurality of spheroids derived from neural progenitor cells and methods of making the spheroids. Also disclosed herein are methods of treating a neuronal disorder or disease.

### INTRODUCTION

Cell populations that retain the ability to differentiate into numerous specialized cell types, such as stem cells, are useful for developing large numbers of lineage specific differentiated cell populations. These differentiated cell populations may include neural progenitor cells (NPCs) that can proliferate or differentiate into neurons, oligodendrocytes, or astrocytes. These lineage specific differentiated cell populations are used in cell replacement therapies for patients with diseases that include a loss or loss of function of a specific cell population. In addition to therapeutic uses, lineage specific differentiated cells are also valuable research tools for a variety of purposes including in vitro screening assays.

Embryonic and somatic stem cells as well as progenitor cells have been used as therapeutics and model systems for neurodegenerative diseases such as for Huntington's, Alzheimer's, Parkinson's, and multiple sclerosis. However, when transplanted in vivo, these cells have poor survival rates, poor recovery of neuronal function, and often result in growth of unwanted tumors. For example, currently survival of progenitor cell transplants is only 5-20% of total cells that were transplanted in vivo. In part due to the poor survival of these cells in vivo, large numbers of cells are required to be transplanted and these transplants often result in inflammation and poor engraftment of the cells.

Therefore, there is a need for compositions and methods to obtain progenitor cell populations capable of being used in vivo with high survival rates for treating diseases that include a loss or loss of function of a specific cell population.

### SUMMARY

In an aspect, the disclosure relates to a stable live-cell composition comprising a plurality of spheroids derived from pluripotent stem cell-derived neural progenitor cells and a pharmaceutical carrier, wherein the spheroids have a diameter of from about 30 μm to about 150 μm. In an embodiment, the pharmaceutical carrier comprises a sugar, sodium hydroxide, and potassium hydroxide. In another embodiment, the sugar is sucrose. In another embodiment, the neural progenitor cells of the spheroids have at least 80% viability for at least 3 days at from about 2° C. to about 8° C. In another embodiment, the neural progenitor cells of the spheroids have at least 80% viability for up to 8 days at from about 2° C. to about 8° C. In another embodiment, the neural progenitor cells of the spheroids have potential to differentiate into mature neurons after being held for at least 3 days in the pharmaceutical carrier at from about 2° C. to about 8° C. In another embodiment, the neural progenitor cells of the spheroids have potential to differentiate into mature neurons after being held for up to 8 days in the pharmaceutical carrier at from about 2° C. to about 8° C. In another embodiment, the spheroids have a cell density of from about 35,000 cells/μL to about 150,000 cells/μL of the pharmaceutical carrier. In another embodiment, the neural progenitor cells express one or more of LMX1a, OTX2, and FOXA2. In another embodiment, the neural progenitor cells do not express one or more of OCT4, NANOG, ESRG, and PAX6.

In a further aspect, the disclosure relates to a method of culturing pluripotent stem cell-derived neural progenitor cells to form spheroids, the method comprising: culturing neural progenitor cells in a first vessel comprising an expansion media to form a plurality of expanded neural progenitor cells; dissociating the expanded neural progenitor cells into single cells; loading the single cells into a second vessel comprising the expansion media, wherein the single cells in the expansion media have a cell density of from about 0.5 million cells/mL to about 1.5 million cells/mL; incubating the single cells in the second vessel for from about 18 hours to about 24 hours; and centrifuging the second vessel at about 7 g to form the spheroids. In an embodiment, the method further comprises: centrifuging the spheroids at from about 50 g to about 200 g for from about 4 minutes to about 10 minutes; discarding supernatant comprising the expansion media; resuspending and incubating the 3D spheroids in a pharmaceutical carrier in a third vessel; removing a portion of supernatant comprising single cells; and resuspending the spheroids in the third vessel. In another embodiment, the spheroids are incubated for about 30 minutes at from about 20° C. to about 25° C. In another embodiment, the spheroids have a cell density of from about 35,000 cells/μL to about 150,000 cells/μL of the pharmaceutical carrier. In another embodiment, the neural progenitor cells of the spheroids have at least 80% viability for at least 3 days at from about 2° C. to about 8° C. In another embodiment, the neural progenitor cells of the spheroids have at least 80% viability for up to 8 days at from about 2° C. to about 8° C. In another embodiment, the neural progenitor cells of the spheroids have potential to differentiate into mature neurons after being held for at least 3 days in the pharmaceutical carrier at from about 2° C. to about 8° C. In another embodiment, the neural progenitor cells of the spheroids have potential to differentiate into mature neurons after being held for up to 8 days in the pharmaceutical carrier at from about 2° C. to about 8° C. In another embodiment, the neural progenitor cells are basal ganglia progenitor cells. In another embodiment, the spheroids have a diameter of from about 30 μm to about 150 μm.

Another aspect of the disclosure provides a method of treating a neuronal disease or disorder, the method comprising administering one or more spheroids derived from stem cell-derived neural progenitor cells into a subject. In an embodiment, the neuronal disease or disorder is one or more of Parkinson's disease, Huntington's disease, stroke, spinal cord injury, epilepsy, traumatic brain injury, cerebral palsy, spasticity, learning and memory, and dystonia. In another embodiment, the neural progenitor cells of the spheroids have a cell survival rate of at least 80% after at least 30 days following administration to the subject. In another embodiment, the neural progenitor cells of the spheroids have a cell survival rate of at least 70% after at least 6 months following administration to the subject. In another embodiment, the spheroids are administered to the subject at a location in need of neuronal cells. In another embodiment, the spheroids are administered by injection. In another embodiment, the spheroids have a diameter of from about 30 μm to about 150 μm. In another embodiment, the spheroids are suspended in a pharmaceutical carrier. In another embodiment, the spheroids have a cell density of from about 35,000 cells/μL to about 150,000 cells/μL of the pharmaceutical carrier.

The disclosure provides for other aspects and embodiments that will be apparent in light of the following detailed description and accompanying figures.

### BRIEF DESCRIPTION OF THE DRAWINGS

FIGS. 1A-1B show quantification of the size of spheroids and high frequency distribution from 40 μm to 60 μm. FIG. 1A is a representative image of plated spheroids for size and number quantification. FIG. 1B is a size frequency distribution bar graph showing that the majority of the spheroids produced with a method described herein range from 20 μm to 180 μm.

FIGS. 2A-2C show that spheroids maintain viability and differential potential into mature neurons up to eight days post-formulation. FIG. 2A is a graph showing spheroid viability analysis measured across ten different spheroid preparations and shows that there was over 80% viability through all the preparations at day 8 post formulation. FIG. 2B is images of MAP2 (green) and Tyrosine Hydroxylase (TH) (red) immunolabeling showing that the spheroids plated at day 3, day 6, and day 8 post-formulation are able to differentiate into mature neurons. FIG. 2C is a graph showing the quantification of immunolabeling of the spheroids of FIG. 2B.

FIGS. 3A-3F shows increased transplanted neuronal progenitor cell survival using spheroid formulation. FIG. 3A is a representative image from a rat brain that received transplanted spheroids showing robust survival of immunolabeled human nuclear antigen+ (HNA+) neuronal progenitors. FIG. 3B is a 100× image from graft border in FIG. 3A showing that the majority of transplanted cells were healthy and only a few were apoptotic (arrows). FIG. 3C is a representative image from a rat brain that received transplanted single cells showing drastically reduced survival of transplanted HNA+ neuronal progenitors compared to FIG. 3A. FIG. 3D is a 100× image from graft border in FIG. 3C showing that there were few healthy transplanted cells and many apoptotic cells (arrows). FIG. 3E is a graph showing that spheroids grafts resulted in about 3.5-fold more HNA+ transplanted cells. FIG. 3F is a graph showing that spheroids grafts resulted in about 2.5-fold larger graft volume than single cell grafts. For FIGS. 3E-F, n=8/group; ***p<0.001 via student's t-test.

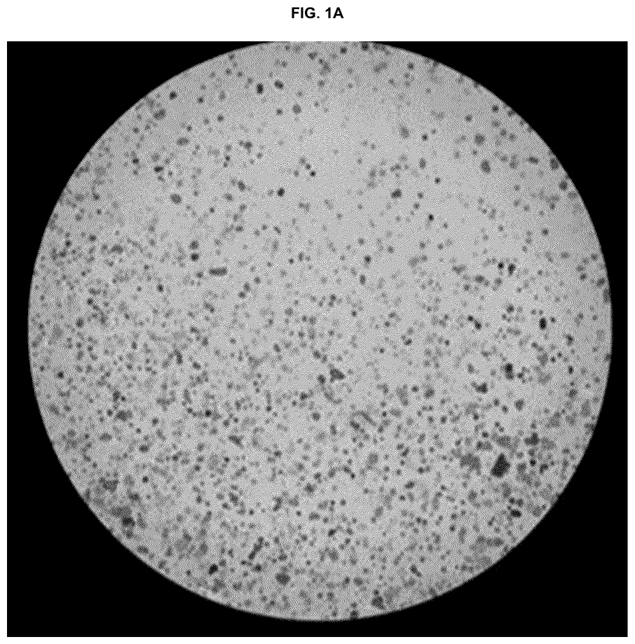

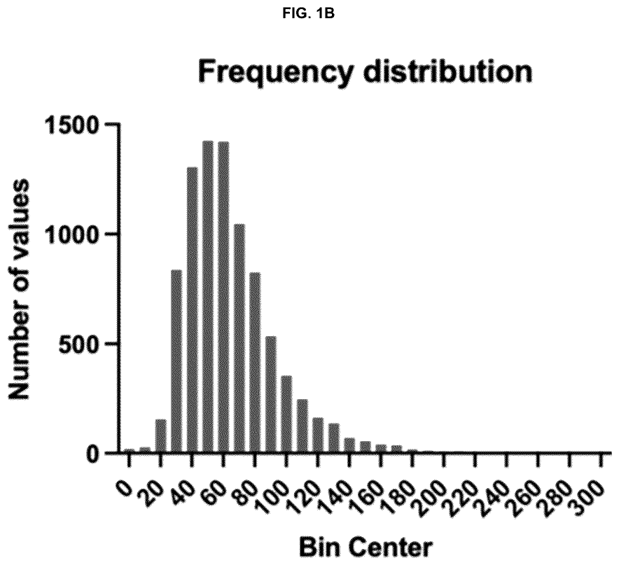

### DETAILED DESCRIPTION

Described herein are compositions of three-dimensional (3D) spheroids made from stem cell-derived neural progenitor cells with diameters ranging in size of 30-150 microns. The spheroid compositions can be stored as live cell preparations for at least 3 days with minimal loss of viability in vitro and the live cell preparations maintain their potential to differentiate to mature neurons. Furthermore, the live cell preparations of spheroids can be directly transplanted into the brain parenchyma and have an overall survival of about 3.5-fold greater when compared to single cell preparations stored in the same conditions. Moreover, there are consistently fewer apoptotic cells observed inside and on the periphery of the grafted spheroids in comparison to the grafted single cells, and the overall graft volumes are considerably greater—about 3 times greater for the spheroids than the same number of total single cells implanted. The spheroid compositions described herein have greater than about 85% total cell survival rates and extensive graft outgrowth in vivo.

The compositions and methods herein have numerous benefits, including but not limited to: the number of total cells needed to be injected into a subject is considerably reduced thus improving safety margins (brain/tissue damage, risk of teratoma, etc.); the volume of injectate is reduced, allowing the use of smaller dimensioned delivery systems; the overall cost of manufacturing is reduced; the compositions described herein can be held and shipped in the live state without the risk of viability loss due to freeze-thaw; reduction of timing for sterility testing (USP sterility requires 14 days) so that sterility is assured before administration to a subject; and, there are less apoptotic cells when used in vivo, which may translate into lower inflammation and better engraftment of the cells.

### 1. DEFINITIONS

Unless otherwise defined, all technical and scientific terms used herein have the same meaning as commonly understood by one of ordinary skill in the art. In case of conflict, the present document, including definitions, will control. Further, unless otherwise required by context, singular terms shall include pluralities and plural terms shall include the singular. Preferred methods and materials are described below, although methods and materials similar or equivalent to those described herein can be used in practice or testing of the present invention. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for cellular activity may be defined in accordance with standard practice. A control may be a subject or cell without spheroids as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Neural cells,” as used herein, refers broadly to cells originating in the central nervous system. Neural cells include, but are not limited to, astrocytes, microglia, oligodendrocytes, and neurons.

By “pluripotency” and “pluripotent” stem cells it is meant that the cells have the ability to differentiate into any type of cell in an organism. The term “induced pluripotent stem cell” or “iPSC” encompasses pluripotent cells, that, like embryonic stem(ES) cells, can be cultured over a long period of time while maintaining the ability to differentiate into any type of cell in an organism. However, unlike ES cells, iPSCs are derived from differentiated somatic cells, that is, cells that had a narrower, more defined potential and that in the absence of experimental manipulation could not give rise to any type of cell in an organism. Human iPSCs (hiPSCs) have a human ES-like morphology, growing as flat colonies with large nucleo-cytoplasmic ratios, defined borders and prominent nuclei. In addition, hiPSCs express several pluripotency markers known by one of ordinary skill in the art, including but not limited to alkaline phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the hiPSCs are capable of forming teratomas. In addition, the hiPSCs are capable of forming or contributing to ectoderm, mesoderm, or endoderm tissues in a living organism.

As used herein, “reprogramming factors” refers to one or more biologically active factors that act on a cell to alter transcription, thereby reprogramming a cell to multipotency or to pluripotency. Reprogramming factors may be provided to cells individually or as a single composition, that is, as a premixed composition, of reprogramming factors. The factors may be provided at the same molar ratio or at different molar ratios. The factors may be provided once or multiple times in the course of culturing the cells. The reprogramming factor may be a combination of transcription factor mRNA's episomal DNAs, viral vectors, including, but not limited to, Oct4, Klf4, Sox2, c-myc, Lin28, and Nanog. Each of the transcription factors may be modified with transcription factor transactivating domains such as GAL1, MyoD, VP16, YAP (Yang, X., et al. Asian Journal of Andrology (2015) 17, 394-402).

“Sample” or “test sample” as used herein can mean any sample comprising spheroids or a component thereof as detailed herein or any sample from a subject to be administered spheroids as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Spheroid” as used herein refers to an artificial construct comprising a plurality of cells, such as neural progenitor cells that have functional properties substantially similar to regions in the brain. Spheroid refers to an aggregate of single cells that form a three-dimensional sphere.

“Stability” as used herein refers to a cell's ability to maintain its characteristics and functions over a prolonged period without significant changes, often indicating a consistent and reliable cell population in research or clinical settings. For example, a stable cell is a cell that remains consistently healthy and functional over time. A “stable” cell or cell line may be comprised of a high percentage of viable cells, indicating good overall health and consistent function. For example, stable neural progenitor cells are a population of neural progenitor cells that can be maintained in culture for extended periods while retaining their ability to self-renew and differentiate into different types of neurons and glial cells, essentially meaning they can consistently produce new neural cells without losing their potential over time and have high viability.

“Stem cells” as used herein have the capacity to undergo self-renewing divisions that produce additional stem cells with the same properties and potential, and divisions that produce daughter cells that differentiate into multiple cell types. Stem cells can be “pluripotent precursor cells” that give rise to all cell types within an organism, or “multipotent precursor cells” that have the capacity to differentiate into a subset of cell types. Embryonic stem cells that are present in the inner cell mass of the blastocyst are an example of pluripotent stem cells. Many types of multipotent stem cells exist and can also be referred to as “progenitor cells.” The embryonic layers and each specific tissue, such as the central nervous system (CNS) tissue, develop from cellular divisions of progenitor cells.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a neurological disease or disorder. The subject may be undergoing other forms of treatment.

“Suspension” as used herein refers to a cell culture system whereby cells are grown in media and do not adhere to any substrate.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Viability” as used herein refers to the state of a cell being alive and functional. For example, viability is a measure of how healthy a cell is. In some instances, viability may be measured by metabolic activity, membrane integrity, and/or the presence of specific markers associated with live cells.

### 2. NEURAL PROGENITOR CELL SPHEROIDS

Provided herein are spheroids that may be derived from neural progenitor cells, adult neural cells, mesenchymal stem cells (MSCs), or neural stem cell lines. In an embodiment, the spheroids may be derived from neural progenitor cells. The spheroids described herein may be used in specific brain regions such as, but not limited to, prefrontal cortex (PFC), nucleus accumbens, amygdala, hippocampus, ventral tegmental area (VTA), by using neural progenitor cells specific for a specific brain region. The spheroids may be in a stable live-cell composition comprising a pharmaceutical carrier.

The spheroids may have a diameter of from about 30 μm to about 150 μm, about 30 μm to about 140 μm, about 30 μm to about 130 μm, about 30 μm to about 120 μm, about 30 μm to about 110 μm, about 30 μm to about 100 μm, about 30 μm to about 90 μm, about 30 μm to about 80 μm, about 30 μm to about 70 μm, about 30 μm to about 60 μm, about 30 μm to about 50 μm, about 30 μm to about 40 μm, about 40 μm to about 150 μm, about 50 μm to about 150 μm, about 60 μm to about 150 μm, about 70 μm to about 150 μm, about 80 μm to about 150 μm, about 90 μm to about 150 μm, about 100 μm to about 150 μm, about 110 μm to about 150 μm, about 120 μm to about 150 μm, about 130 μm to about 150 μm, or about 140 μm to about 150 μm. In an embodiment, the spheroids may have a diameter of from about 30 μm to about 150 μm. In another embodiment, the spheroids may have a diameter of from about 50 μm to about 150 μm.

### a. Neural Progenitor Cells

The neural progenitor cells are progenitor cells of the central nervous system (CNS) that give rise to many, if not all, of the glial and neuronal cell types that populate the CNS. The neural progenitor cells may be derived from pluripotent stem cells. The pluripotent stem cells are able to self-renew by dividing and developing into the three primary groups of cells: ectoderm that gives rise to the skin and nervous system; endoderm that forms the gastrointestinal and respiratory tracts, endocrine glands, liver, and pancreas; and mesoderm that forms bone, cartilage, most of the circulatory system, muscles, connective tissue, and the like. The stem cells may be induced pluripotent cell (iPSCs), conventional embryonic stem cell (ES cells) that are derived from embryos, embryonic stem cells made by somatic cell nuclear transfer (ntES cells), or embryonic stem cells from unfertilized eggs (parthenogenesis embryonic stem cells, or pES cells).

The neural progenitor cells may be capable of giving rise to mature neuronal cells. The mature neuronal cells may be able to express 160 kDa neuro-filament protein, MAP2ab, glutamate, synaptophysin, glutamic acid decarboxylase (GAD), tyrosine hydroxylase, GABA, serotonin, or a combination thereof. The neural progenitor cells may be capable of differentiating into glial cells, including astrocytes and oligodendrocytes. The glial cells may also include microglial cells and radial glial cells.

The neural progenitor cells may be capable of establishing a graft in a recipient brain of a subject in need thereof.

The neural progenitor cells of the spheroids described herein may have at least 60%, at least 70%, at least 80%, or at least 90% viability for at least 2 days, at least 3 days, at least 4 days, at least 5 days, at least 6 days, or at least 7 days at from about 2° C. to about 8° C. The neural progenitor cells of the spheroids described herein may have at least 60%, at least 70%, at least 80%, or at least 90% viability for up to 8 days, up to 9 days, up to 10 days, up to 11 days, up to 12 days, up to 13 days, or up to 14 days at from about 2° C. to about 8° C. In some embodiments, the neural progenitor cells of the spheroids described herein may have at least 80% viability for from about 3 days to about 8 days at about 4° C.

The neural progenitor cells of the spheroids described herein may be stable for at least 2 days, at least 3 days, at least 4 days, at least 5 days, at least 6 days, or at least 7 days at from about 2° C. to about 8° C. The neural progenitor cells of the spheroids described herein may be stable for up to 8 days, up to 9 days, up to 10 days, up to 11 days, up to 12 days, up to 13 days, or up to 14 days at from about 2° C. to about 8° C. In some embodiments, the neural progenitor cells of the spheroids described herein may be stable from about 3 days to about 8 days at about 4° C.

The neural progenitor cells of the spheroids described herein may be stable and have at least 60%, at least 70%, at least 80%, or at least 90% viability for at least 2 days, at least 3 days, at least 4 days, at least 5 days, at least 6 days, or at least 7 days at from about 2° C. to about 8° C. The neural progenitor cells of the spheroids described herein may be stable and have at least 60%, at least 70%, at least 80%, or at least 90% viability for up to 8 days, up to 9 days, up to 10 days, up to 11 days, up to 12 days, up to 13 days, or up to 14 days at from about 2° C. to about 8° C. In some embodiments, the neural progenitor cells of the spheroids described herein may be stable and have at least 80% viability for from about 3 days to about 8 days at about 4° C.

The neural progenitor cells of the spheroids as described herein may have potential to differentiate into mature neurons after being held for at least 2 days, at least 3 days, at least 4 days, at least 5 days, at least 6 days, or at least 7 days in a pharmaceutical carrier at from about 2° C. to about 8° C. The neural progenitor cells of the spheroids as described herein may have potential to differentiate into mature neurons after being held for up to 8 days, up to 9 days, up to 10 days, up to 11 days, up to 12 days, up to 13 days, or up to 14 days in a pharmaceutical carrier at from about 2° C. to about 8° C. In some embodiments, the neural progenitor cells of the spheroids described herein may have potential to differentiate into mature neurons after being held for from about 3 days to about 8 days in a pharmaceutical carrier at about 4° C.

The neural progenitor cells may express one or more of LMX1a, OTX2, and FOXA2. The neural progenitor cells do not express one or more of OCT4, NANOG, ESRG, and PAX6.

The neural progenitor cells may be basal ganglia progenitor cells, including corpus striatum with the caudate and the lenticular nuclei (the putamen, globus pallidus externus, and internus) and the subthalamic nucleus and the substantia nigra, spinal interneuron progenitor cells, cortical progenitor cells, GABA progenitor cells, glutaminergic progenitor cells, dopaminergic progenitor cells, locus ceruleus progenitor cells, serotonergic progenitor cells, hypothalamic progenitor cells, hippocampal progenitor cells, or cerebellar progenitor cells. In some embodiments, the neural progenitor cells may be basal ganglia progenitor cells.

### 3. COMPOSITIONS

Further provided herein are stable live-cell compositions comprising the above-described neural progenitor cell spheroids. In some embodiments, the stable live-cell composition may comprise a plurality of spheroids derived from pluripotent stem cell-derived neural progenitor cells as described herein and a pharmaceutical carrier. The plurality of spheroids may be suspended in the pharmaceutical carrier. The spheroids may have a cell density of from about 30,000 cells/μL to about 150,000 cells/μL, about 35,000 cells/μL to about 150,000 cells/μL, about 50,000 cells/μL to about 150,000 cells/μL, about 65,000 cells/μL to about 150,000 cells/μL, about 80,000 cells/μL to about 150,000 cells/μL, about 95,000 cells/μL to about 150,000 cells/μL, about 110,000 cells/μL to about 150,000 cells/μL, about 125,000 cells/μL to about 150,000 cells/μL, about 140,000 cells/μL to about 150,000 cells/μL, about 30,000 cells/μL to about 170,000 cells/μL, about 30,000 cells/μL to about 155,000 cells/μL, about 30,000 cells/μL to about 140,000 cells/L, about 30,000 cells/μL to about 125,000 cells/μL, about 30,000 cells/μL to about 110,000 cells/μL, about 30,000 cells/μL to about 95,000 cells/μL, about 30,000 cells/μL to about 80,000 cells/μL, about 30,000 cells/μL to about 65,000 cells/L, about 30,000 cells/μL to about 50,000 cells/μL, or about 30,000 cells/μL to about 35,000 cells/μL of the pharmaceutical carrier. In some embodiments, the spheroids may have a cell density of from about 35,000 cells/μL to about 150,000 cells/μL of the pharmaceutical carrier. In a particular embodiment, the spheroids may have a cell density of about 75,000 cells/μL of the pharmaceutical carrier.

The spheroids as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol, and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutical carrier may be a non-toxic, inert solid, semi-solid, liquid filler, diluent, encapsulating material, or formulation auxiliary of any type. Pharmaceutical carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. In an embodiment, the pharmaceutical carrier may comprise a sugar, sodium hydroxide, and potassium hydroxide. The sugar may be sucrose. In a particular embodiment, the pharmaceutical carrier may be HypoThermosol®.

### 4. ADMINISTRATION

The spheroids as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed spheroids, or compositions comprising the same, may be administered to a subject by different routes including intracranial, intrathecal, parenterally, sublingually, transdermally, transmucosally, intrapleurally, intravenous, intraarterial, intraperitoneal, subcutaneous, intradermally, epidermally, intramuscular, intraarticular, or combinations thereof.

The spheroids as detailed herein, or the pharmaceutical compositions comprising the same, may be injected into the brain or other component of the CNS. Injection may be conducted in any manner so as to introduce the spheroids into the nervous system of a subject. Preferably, the spheroids are introduced into a specific site in the nervous system. Any method may be used to introduce the cells into a specific location. The spheroids may be injected using a micro-glass pipette (for example, having a 300-micron outer diameter) connected to a micro-injector. The glass pipette may be modified to limit the depth of penetration into the subject's nervous system. The spheroids may be also injected by a Hamilton syringe into predetermined depth using a stereotaxic device. Any stereotaxic injection method may be suitable. The volume that is injected and the concentration of spheroids in the transplanted composition may depend on the indication for transplantation, the location in the nervous system, and the species of the subject.

For veterinary use, the spheroids, or compositions comprising the same, may be administered as a suitably acceptable formulation in accordance with normal veterinary practice. The veterinarian may readily determine the dosing regimen and route of administration that is most appropriate for a particular animal.

Upon delivery of the presently disclosed spheroids, or the pharmaceutical compositions comprising the same, and thereupon the neural progenitor cells of the spheroids into the nervous system of the subject, the administered neural progenitor cells may differentiate into mature neurons.

When the neural progenitor cells of the spheroids are engrafted into a developing nervous system of a subject, the progenitor cells may participate in the processes of normal development and may respond to the subject's developmental cues. The engrafted progenitor cells may migrate along established migratory pathways and may spread widely into disseminated areas of the nervous system. The transplanted neural progenitor cells may respond to the subject's environmental signals, differentiate in a temporally and regionally appropriate manner into progeny from both the neuronal and glial lineages in accord to the region's stage of development and in concert with the subject's developmental program. The engrafted neural progenitor cells may be capable of non-disruptive intermingling with the host neural progenitors as well as differentiated cells. The transplanted neural progenitor cells can replace specific deficient neuronal or glial cell populations, restore defective functions, and can express foreign genes in a wide distribution.

The spheroids derived from neural progenitor cells may be transplanted into a developed nervous system of a subject. The neural progenitor cells can form a stable graft, migrate within the subject's nervous system, intermingle, and interact with the subject's neural progenitors and differentiated cells. The neural progenitor cells can replace specific deficient neuronal or glial cell populations, restore deficient functions, and activate regenerative and healing processes in the subject's nervous system. The transplanted neural progenitor cells can express foreign genes in the subject's nervous system.

### 5. METHODS

### a. Methods of Forming Spheroids

Provided herein are methods of culturing stem cell-derived neural progenitor cells to form spheroids as described herein. The methods may include culturing neural progenitor cells in a first vessel comprising an expansion media to form a plurality of expanded neural progenitor cells; dissociating the expanded neural progenitor cells into single cells; loading the single cells into a second vessel comprising the expansion media; incubating the single cells in the second vessel; and centrifuging the second vessel to form the spheroids.

The single cells in the expansion media may have a cell density of from about 0.5 million cells/mL to about 1.5 million cells/mL, about 1.0 million cells/mL to about 1.5 million cells/mL, or about 0.5 million cells/mL to about 1.0 million cells/mL. In some embodiments, the expansion media may have a cell density of about 1.0 million cells/mL.

The single cells may be incubated in the second vessel for from about 18 hours to about 24 hours, about 20 hours to about 24 hours, about 22 hours to about 24 hours, about 18 hours to about 22 hours, or about 18 hours to about 20 hours. In some embodiments, the single cells may be incubated in the second vessel for about 24 hours. The spheroids may be incubated for about 20 minutes, about 25 minutes, about 30 minutes, about 35 minutes, or about 40 minutes at from about 20° C. to about 25° C., about 21° C. to about 25° C., about 22° C. to about 25° C., about 23° C. to about 25° C., about 24° C. to about 25° C., about 20° C. to about 24° C., about 20° C. to about 23° C., about 20° C. to about 22° C., or about 20° C. to about 21° C. In some embodiments, the spheroids may be incubated for about 30 minutes at from about 20° C. to about 25° C.

The second vessel may be centrifuged at about at from about 5 g to about 15 g, from about 6 g to about 15 g, from about 7 g to about 15 g, from about 8 g to about 15 g, from about 9 g to about 15 g, from about 10 g to about 15 g, from about 11 g to about 15 g, from about 12 g to about 15 g, from about 13 g to about 15 g, from about 14 g to about 15 g, from about 5 g to about 14 g, from about 5 g to about 13 g, from about 5 g to about 12 g, from about 5 g to about 11 g, from about 5 g to about 10 g, from about 5 g to about 9 g, from about 5 g to about 8 g, from about 5 g to about 7 g, or from about 5 g to about 6 g. The second vessel may be centrifuged at about at about 5 g, about 6 g, about 7 g, about 8 g, about 9 g, about 10 g, about 11 g, about 12 g, about 13 g, about 14 g, or about 15 g. In an embodiment, the second vessel may be centrifuged at about at about 7 g.

The method may further comprise centrifuging the spheroids at from about 50 g to about 200 g, about 100 g to about 200 g, about 150 g to about 200 g, about 50 g to about 150 g, or about 50 g to about 100 g for from about 4 minutes to about 10 minutes, about 6 minutes to about 10 minutes, about 8 minutes to about 10 minutes, about 4 minutes to about 8 minutes, or about 4 minutes to about 6 minutes. In some embodiments, the method may further comprise centrifuging the spheroids at about 100 g for about 5 minutes.

The method may further comprise discarding supernatant comprising the expansion media; resuspending and incubating the 3D spheroids in a pharmaceutical carrier in a third vessel; removing a portion of supernatant comprising single cells; and resuspending the spheroids in the third vessel.

### b. Methods of Treating a Neuronal Disease or Disorder

Provided herein are methods of treating a neuronal disease or disorder. The methods may include administering one or more spheroids derived from stem cell-derived neural progenitor cells as described herein into a subject. The neuronal disease or disorder may be one or more of Parkinson's disease, Huntington's disease, stroke, spinal cord injury, epilepsy, traumatic brain injury, cerebral palsy, spasticity, learning and memory, and dystonia. The spheroids may be administered to the subject at a location in need of neuronal cells. The spheroids may be administered to the subject by injection.

The neural progenitor cells of the spheroids may have a cell survival rate of at least 50%, at least 60%, at least 70%, at least 80%, or at least 90% after at least 20 days, at least 25 days, at least 30 days, at least 1 month, at least 2 months, at least 3 months, at least 4 months, at least 5 months, at least 6 months, at least 7 months, at least 8 months, at least 9 months, at least 10 months, at least 11 months, or at least 12 months following administration to the subject. In some embodiments, the neural progenitor cells of the spheroids may have a cell survival rate of at least 80% after at least 30 days following administration to the subject. In another embodiment, the neural progenitor cells of the spheroids may have a cell survival rate of at least 70% after at least 6 months following administration to the subject.

### 6. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Spheroids and single cells formulation. Progenitor cells were thawed and expanded for seven days in expansion media (DMEM/F12 CTS, N2 CTS, Glutamax® CTS (Gibco, Waltham, MA), ascorbic acid (Sigma, Burlington, MA), HEPES Buffer (Lonza, Basel, Switzerland)). On day seven the cells were detached and dissociated into single cells using Accutase™ (Sigma, Burlington, MA) and loaded into PBS mini vessel (PBS Biotech, Camarillo, CA) at a density of 1 million cells/mL in a total volume of 60 mL expansion media for spheroids formulation or resuspended at a final cell density of 75,000 cells/μL and stored in Hypo Thermosol® at 4° C. for single cells preparation. The cells were formulated into spheroids after a 24-hour incubation in the PBS mini vessel at 60 rpm. Post formulation the spheroids were removed from the PBS mini vessel, and centrifuged at 100 g for 5 min. The supernatant was discarded completely and the spheroids were resuspended in 1.5 mL of Hypo Thermosol® transferred into an 1.8 mL Eppendorf tube, and left for 30 minutes at room temperature (i.e. about 20° C. to about 25° C.) to deposit. After 30 minutes, most of the spheroids were at the bottom of the tube and the single cells found at the surface of the tube were discarded by removing 1.2 mL of the supernatant. The spheroids in the remaining 300 μL were gently resuspended and quantified for cell density and spheroids number using trypsin to dissociate them into single cells and image-based quantification. The spheroids preparation was adjusted to a final cell density of 75,000 cells/μL and stored at 4° C. for up to eight days.

Spheroids viability and maintenance of the differentiation potential analysis. Spheroids viability was assessed daily for up 8 days post formulation by single cell dissociation after 5 minutes of incubation at 37° C. in trypsin using the MOXY V cell count. The viability was registered and plotted using GraphPad Prism 9.

Spheroids were plated at post formulation days 3, 6, and 8 at 1.5 million cells/6 well plate for expansion and then differentiated to mature tyrosine hydroxylase (TH) positive dopaminergic neurons following published protocols (Xiong et al., Cell Stem Cell (2020) 28(1): P112-126; Xi et al., Stem Cells. (2012) 30(8):1655-1663). Cells were immunolabeled for TH and MAP2 at differentiation day 14 and imaged and analyzed using Keyence microscopes.

Cell transplantation and stereological cell count analysis. Cells were 300,000 human neuronal progenitors in Hypo Thermosol® vehicle were stereotactically transplanted into male immunodeficient rat brains in either a single cell (n =8) or spheroids (n =8) formulation. 30 days post-transplant, rats were sacrificed, and brains were fixed, extracted, sectioned in the coronal plane through the transplanted region and immunolabeled for human nuclear antigen (HNA) to visualize transplanted cells. Using Image J software (NIH) and plugins Grid, Counts and Grid 2, unbiased stereological cell counts were performed via the optical fractionator method (Ip et al., J Vis Exp. 2017; (127): 56103) on 100× magnification images from 8 coronal sections/brain spaced 200 μm apart. Graft volumes were quantified with Image J on 4× magnification images via Cavalieri estimation (Pakkenberg et al., J Neurol Neurosurg Psychiatry. 1991; 54(1):30-3).

### Example 2

### Spheroids Maintain Viability and Differential Potential Into Mature Neurons Up to Eight Days Post-Formulation

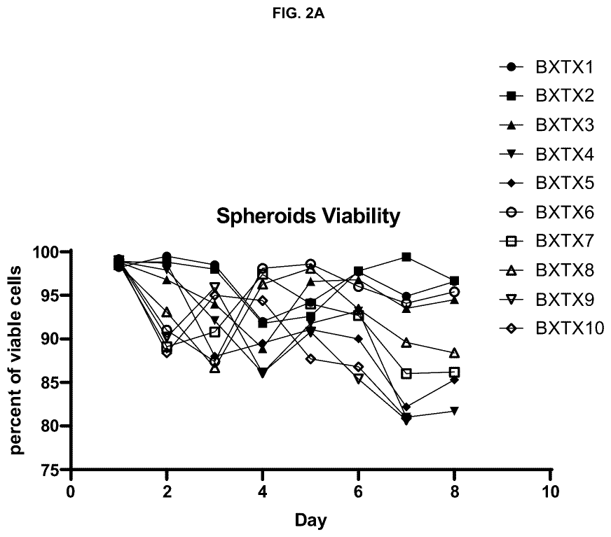

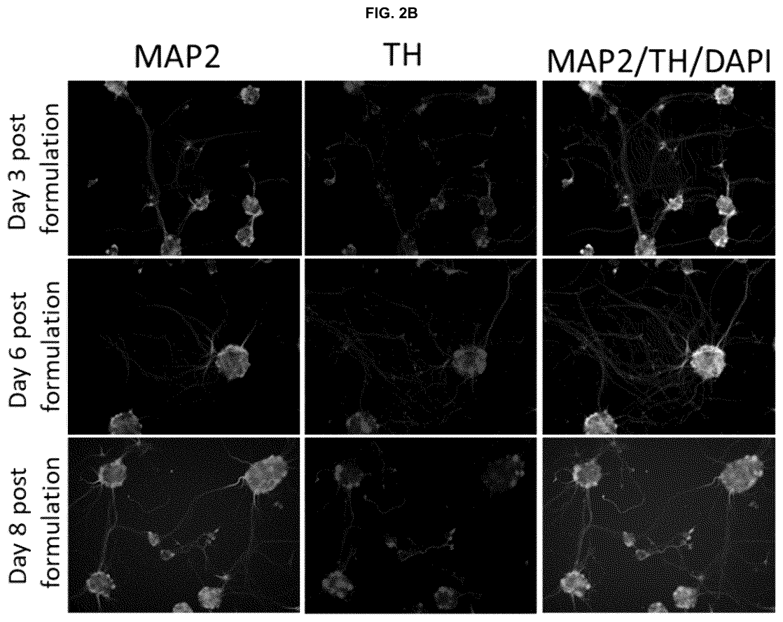

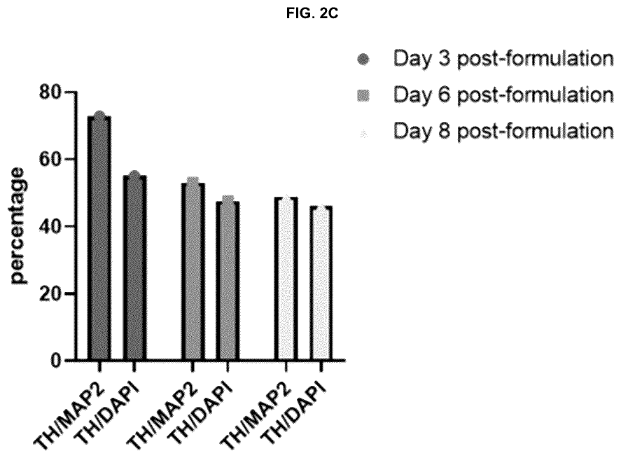

Stem cell-derived neural progenitor cells were cultured into 3D spheroids using a PBS bioreactor or the like. The 3D spheroids had diameters ranging in size of about 50-150 microns. Upon formation of the 3D spheroids, the spheroids were resuspended into a non-frozen storage and shipping stability storage medium called Hypothermosol® (BioLife Solutions, Bothell, WA). The spheroid preparations can be stored as live cell preparations for up to 8 days with minimal loss in viability in vitro and maintain their potential to differentiate into mature neurons (FIGS. 2A-2C). FIG. 2A shows that the 10 different cell lines used to make spheroids had over 80% viability at day 8 post formulation. FIG. 2B and FIG. 2C show that the spheroids plated at day 3, day 6, and day 8 post-formulation were able to differentiate into mature neurons.

### Example 3

### Increased Transplanted Neuronal Progenitor Cell Survival Using Spheroid Formulation

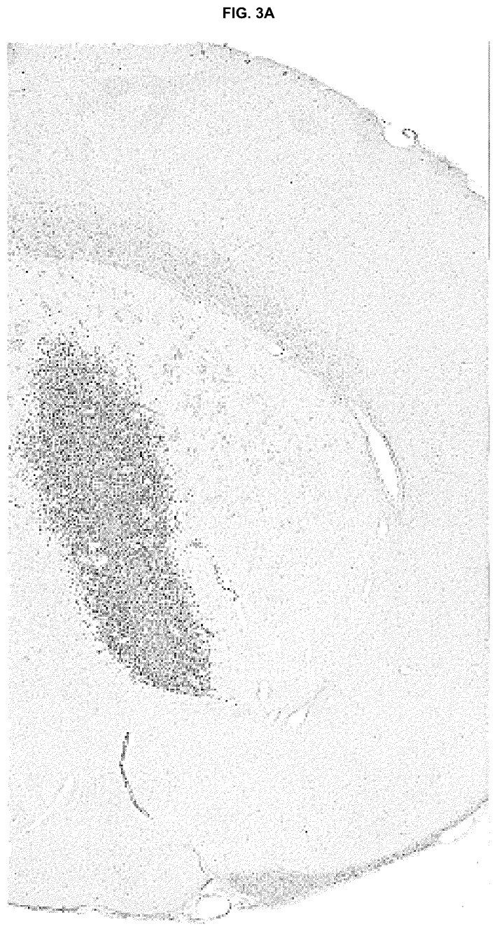

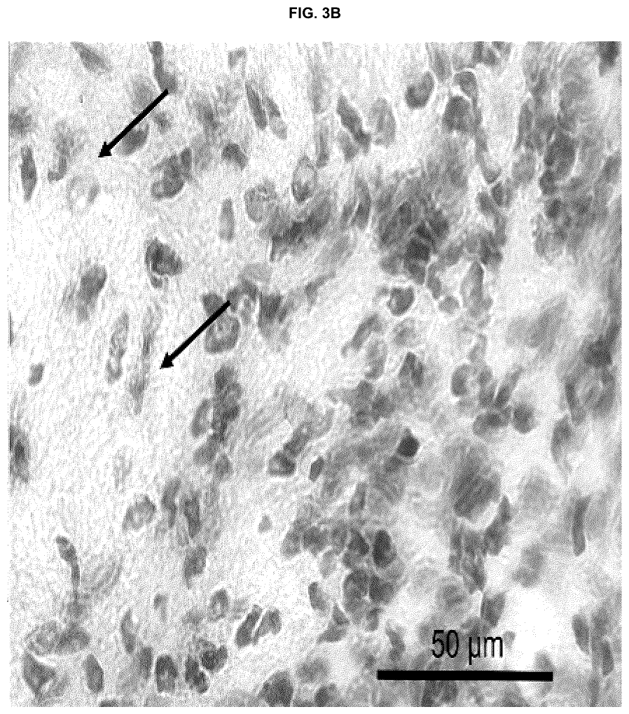

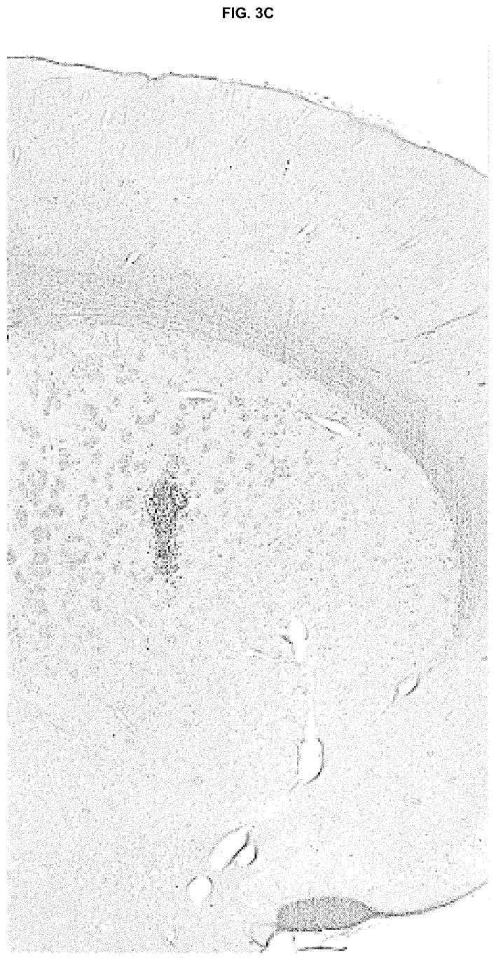

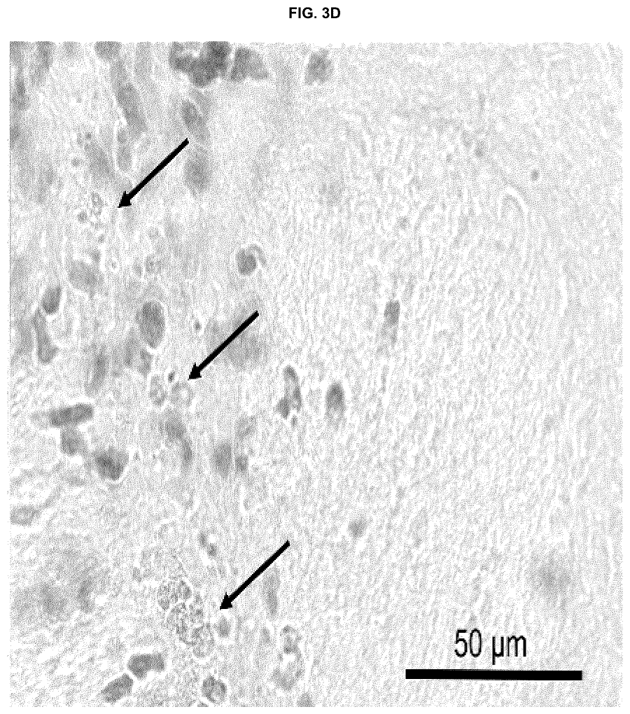

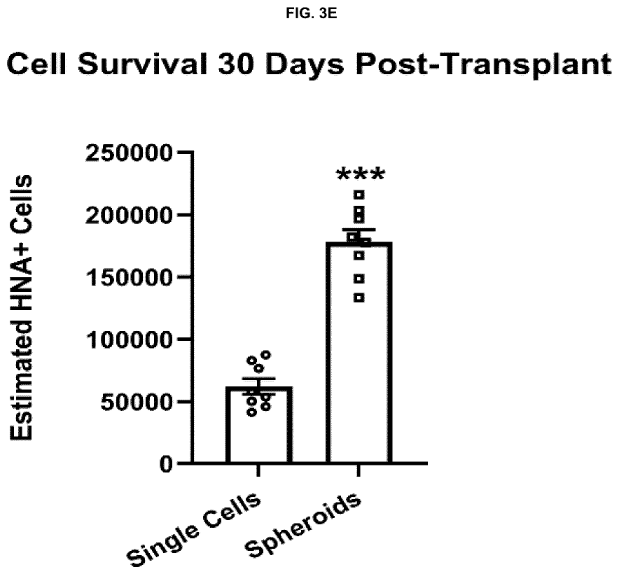

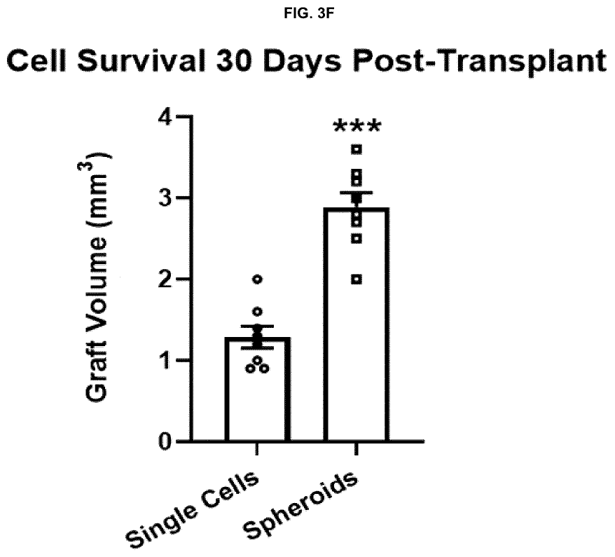

When the spheroids were stored live for up to 48 hours and were directly transplanted into the brain parenchyma, their overall survival was 3.5-fold greater than single cell preparations transplanted and stored in the same conditions (FIGS. 3A-3F). Moreover, there were consistently fewer apoptotic cells observed inside and on the periphery of the grafted spheroids in comparison to the grafted single cells (FIG. 3D). In addition, the overall graft volumes were considerably greater, the overall graft volume was about 3 times greater in the spheroid preparations for the same number of total single cells implanted (FIG. 3F). Currently survival of progenitor cell transplants ranges from 5-20% of total cells when using single cells. Here, it has been demonstrated that the use of spheroids results in a survival rate that is greater than 85% of the total cell in addition to extensive graft outgrowth in vivo.

These data indicate that the number of total cells needed to be injected can be considerably reduced when using spheroids, thus improving safety margins (brain/tissue damage, risk of teratoma etc.); the volume of injectate can be reduced when using spheroids, allowing the use of smaller dimensioned delivery systems; the spheroids can be held and shipped in the live state without the risk of viability loss due to freeze thaw; and, less apoptotic cells when using spheroids may translate into lower inflammation and better engraftment of the spheroids as compared to single cells.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A stable live-cell composition comprising a plurality of spheroids derived from pluripotent stem cell-derived neural progenitor cells and a pharmaceutical carrier, wherein the spheroids have a diameter of from about 30 μm to about 150 μm.

Clause 2. The composition of clause 1, wherein the pharmaceutical carrier comprises a sugar, sodium hydroxide, and potassium hydroxide.

Clause 3. The composition of clause 3, wherein the sugar is sucrose.

Clause 4. The composition of any one of clauses 1-3, wherein the neural progenitor cells of the spheroids have at least 80% viability for at least 3 days at from about 2° C. to about 8° C.

Clause 5. The composition of any one of clauses 1-4, wherein the neural progenitor cells of the spheroids have at least 80% viability for up to 8 days at from about 2° C. to about 8° C.

Clause 6. The composition of any one of clauses 1-5, wherein the neural progenitor cells of the spheroids have potential to differentiate into mature neurons after being held for at least 3 days in the pharmaceutical carrier at from about 2° C. to about 8° C.

Clause 7. The composition of any one of clauses 1-6, wherein the neural progenitor cells of the spheroids have potential to differentiate into mature neurons after being held for up to 8 days in the pharmaceutical carrier at from about 2° C. to about 8° C.

Clause 8. The composition of any one of clauses 1-7, wherein the spheroids have a cell density of from about 35,000 cells/μL to about 150,000 cells/μL of the pharmaceutical carrier.

Clause 9. The composition of any one of clauses 1-8, wherein the neural progenitor cells express one or more of LMX1a, OTX2, and FOXA2.

Clause 10. The composition of any one of clauses 1-9, wherein the neural progenitor cells do not express one or more of OCT4, NANOG, ESRG, and PAX6.

Clause 11. A method of culturing pluripotent stem cell-derived neural progenitor cells to form spheroids, the method comprising: culturing neural progenitor cells in a first vessel comprising an expansion media to form a plurality of expanded neural progenitor cells; dissociating the expanded neural progenitor cells into single cells; loading the single cells into a second vessel comprising the expansion media, wherein the single cells in the expansion media have a cell density of from about 0.5 million cells/mL to about 1.5 million cells/mL; incubating the single cells in the second vessel for from about 18 hours to about 24 hours; and centrifuging the second vessel at about 7 g to form the spheroids.

Clause 12. The method of clause 11, wherein the method further comprises: centrifuging the spheroids at from about 50 g to about 200 g for from about 4 minutes to about 10 minutes; discarding supernatant comprising the expansion media; resuspending and incubating the 3D spheroids in a pharmaceutical carrier in a third vessel; removing a portion of supernatant comprising single cells; and resuspending the spheroids in the third vessel.

Clause 13. The method of clause 12, wherein the spheroids are incubated for about 30 minutes at from about 20° C. to about 25° C.

Clause 14. The method of clause 12 or clause 13, wherein the spheroids have a cell density of from about 35,000 cells/μL to about 150,000 cells/μL of the pharmaceutical carrier.

Clause 15. The method of any one of clauses 11-14, wherein the neural progenitor cells of the spheroids have at least 80% viability for at least 3 days at from about 2° C. to about 8° C.

Clause 16. The method of any one of clauses 11-15, wherein the neural progenitor cells of the spheroids have at least 80% viability for up to 8 days at from about 2° C. to about 8° C.

Clause 17. The method of any one of clauses 11-16, wherein the neural progenitor cells of the spheroids have potential to differentiate into mature neurons after being held for at least 3 days in the pharmaceutical carrier at from about 2° C. to about 8° C.

Clause 18. The method of any one of clauses 11-17, wherein the neural progenitor cells of the spheroids have potential to differentiate into mature neurons after being held for up to 8 days in the pharmaceutical carrier at from about 2° C. to about 8° C.

Clause 19. The method of any one of clauses 11-18, wherein the neural progenitor cells are basal ganglia progenitor cells.

Clause 20. The method of any one of clauses 11-19, wherein the spheroids have a diameter of from about 30 μm to about 150 μm.

Clause 21. A method of treating a neuronal disease or disorder, the method comprising administering one or more spheroids derived from stem cell-derived neural progenitor cells into a subject.

Clause 22. The method of clause 21, wherein the neuronal disease or disorder is one or more of Parkinson's disease, Huntington's disease, stroke, spinal cord injury, epilepsy, traumatic brain injury, cerebral palsy, spasticity, learning and memory, and dystonia.

Clause 23. The method of clause 21 or clause 22, wherein the neural progenitor cells of the spheroids have a cell survival rate of at least 80% after at least 30 days following administration to the subject.

Clause 24. The method of any one of clauses 21-23, wherein the neural progenitor cells of the spheroids have a cell survival rate of at least 70% after at least 6 months following administration to the subject.

Clause 25. The method of any one of clauses 22-24, wherein the spheroids are administered to the subject at a location in need of neuronal cells.

Clause 26. The method of any one of clauses 22-25, wherein the spheroids are administered by injection.

Clause 27. The method of any one of clauses 22-26, wherein the spheroids have a diameter of from about 30 μm to about 150 μm.

Clause 28. The method of any one of clauses 22-27, wherein the spheroids are suspended in a pharmaceutical carrier.

Clause 29. The method of clause 28, wherein the spheroids have a cell density of from about 35,000 cells/μL to about 150,000 cells/μL of the pharmaceutical carrier.

1. A stable live-cell composition comprising a plurality of spheroids derived from pluripotent stem cell-derived neural progenitor cells and a pharmaceutical carrier, wherein the spheroids have a diameter of from about 30 μm to about 150 μm.

2. The composition of claim 1, wherein the pharmaceutical carrier comprises a sugar, sodium hydroxide, and potassium hydroxide.

3. The composition of claim 3, wherein the sugar is sucrose.

4. The composition of claim 1, wherein the neural progenitor cells of the spheroids have at least 80% viability for at least 3 days at from about 2° C. to about 8° C.

5. (canceled)

6. The composition of claim 1, wherein the neural progenitor cells of the spheroids have potential to differentiate into mature neurons after being held for at least 3 days in the pharmaceutical carrier at from about 2° C. to about 8° C.

7. (canceled)

8. The composition of claim 1, wherein the spheroids have a cell density of from about 35,000 cells/μL to about 150,000 cells/μL of the pharmaceutical carrier.

9. The composition of claim 1, wherein the neural progenitor cells express one or more of LMX1a, OTX2, and FOXA2; and/or
wherein the neural progenitor cells do not express one or more of OCT4, NANOG, ESRG, and PAX6.

10. (canceled)

11. A method of culturing pluripotent stem cell-derived neural progenitor cells to form spheroids, the method comprising:
culturing neural progenitor cells in a first vessel comprising an expansion media to form a plurality of expanded neural progenitor cells;
dissociating the expanded neural progenitor cells into single cells;
loading the single cells into a second vessel comprising the expansion media, wherein the single cells in the expansion media have a cell density of from about 0.5 million cells/mL to about 1.5 million cells/mL;
incubating the single cells in the second vessel for from about 18 hours to about 24 hours; and
centrifuging the second vessel at about 7 g to form the spheroids.

12. The method of claim 11, wherein the method further comprises:
centrifuging the spheroids at from about 50 g to about 200 g for from about 4 minutes to about 10 minutes;
discarding supernatant comprising the expansion media;
resuspending and incubating the 3D spheroids in a pharmaceutical carrier in a third vessel;
removing a portion of supernatant comprising single cells; and
resuspending the spheroids in the third vessel.

13. The method of claim 12, wherein the spheroids are incubated for about 30 minutes at from about 20° C. to about 25° C.

14. The method of claim 12, wherein the spheroids have a cell density of from about 35,000 cells/μL to about 150,000 cells/μL of the pharmaceutical carrier.

15. The method of claim 11, wherein the neural progenitor cells of the spheroids have at least 80% viability for at least 3 days at from about 2° C. to about 8° C.

16. (canceled)

17. The method of claim 11, wherein the neural progenitor cells of the spheroids have potential to differentiate into mature neurons after being held for at least 3 days in the pharmaceutical carrier at from about 2° C. to about 8° C.

18. (canceled)

19. The method of claim 11, wherein the neural progenitor cells are basal ganglia progenitor cells.

20. The method of claim 11, wherein the spheroids have a diameter of from about 30 μm to about 150 μm.

21. A method of treating a neuronal disease or disorder, the method comprising administering one or more spheroids derived from stem cell-derived neural progenitor cells into a subject.

22. The method of claim 21, wherein the neuronal disease or disorder is one or more of Parkinson's disease, Huntington's disease, stroke, spinal cord injury, epilepsy, traumatic brain injury, cerebral palsy, spasticity, learning and memory, and dystonia.

23. The method of claim 21, wherein the neural progenitor cells of the spheroids have a cell survival rate of at least 80% after at least 30 days following administration to the subject.

24. The method of claim 21, wherein the neural progenitor cells of the spheroids have a cell survival rate of at least 70% after at least 6 months following administration to the subject.

25. The method of claim 22, wherein the spheroids are administered to the subject at a location in need of neuronal cells.

26. The method of claim 22, wherein the spheroids are administered by injection.

27. The method of claim 22, wherein the spheroids have a diameter of from about 30 μm to about 150 μm.

28. The method of claim 22, wherein the spheroids are suspended in a pharmaceutical carrier.

29. The method of claim 28, wherein the spheroids have a cell density of from about 35,000 cells/μL to about 150,000 cells/μL of the pharmaceutical carrier.
